(19) United States
(12) Patent Application Publication
(10) Pub. No.: US 2026/0054036 A1
(43) Pub. Date: Feb. 26, 2026

(54) Integrated Safety IV Catheter System with Port for Attachment of Blood Collection Device

(71) Applicant: Becton, Dickinson and Company, Franklin Lakes, NJ (US)

(72) Inventors: Jonathan Karl Burkholz, Salt Lake City, UT (US); Mukilan Balamurugan, Theni (IN); Edwin Jeyaseelan Arockia Doss, Madurai (IN); Balaji Kannan, Chennai (IN)

(21) Appl. No.: 19/196,021

(22) Filed: May 1, 2025

Related U.S. Application Data
(60) Provisional application No. 63/642,083, filed on May 3, 2024.

(51) Int. Cl. A61M 25/06; A61B 5/15; A61M 39/00; A61M 39/10; A61M 39/28

(52) U.S. Cl. CPC A61M 25/0606; A61B 5/15003; A61B 5/150206; A61M 25/0637; A61M 39/10; A61M 39/28; A61M 2039/0009; A61M 2039/1027; A61M 2039/1072; A61M 2039/1077; A61M 2202/0014; A61M 2202/0413; A61M 2205/3331

(57) ABSTRACT

A catheter system is provided that includes a catheter and a first catheter adapter having an adapter body and an adapter wing. The catheter body includes distal, proximal and side ports thereon, with the catheter proximal end secured to the distal port and the side port comprising a luer connection. The adapter wing is joined to the adapter body and includes first and second flexible wings configured to be brought into contact with each other when a pinching force is applied thereto. An extension tube is connected to the proximal port and a second catheter adapter is connected to the proximal end of the extension tube. A blood draw device is coupled to the side port and includes a flow tube configured to be advanced distally out from the blood draw device and through the first catheter adapter and into the catheter to perform a blood draw.

### CROSS-REFERENCE TO RELATED APPLICATION

The present application claims priority to U.S. Provisional Patent Application No. 63/642,083 entitled “Integrated Safety IV Catheter System with Port for Attachment of Blood Collection Device” filed May 3, 2024, the disclosure of which is hereby incorporated by reference in its entirety.

### BACKGROUND OF THE INVENTION

### Field of the Invention

The present invention is directed to a safety IV catheter system used for insertion of an indwelling catheter.

### Background of the Invention

Vascular access devices (VADs) are used in the medical field to access peripheral vasculature of a patient for purposes of infusion therapy and/or blood withdrawal. A common type of VAD is an over-the-needle peripheral intravenous catheter (PIVC). As its name implies, the “over-the-needle” PIVC may be mounted over an introducer needle having a sharp distal tip. The sharp distal tip may be used to pierce skin and the vasculature of the patient. Insertion of the PIVC into the vasculature may follow the piercing of the vasculature by the needle. The needle and the PIVC are generally inserted at a shallow angle through the skin into the vasculature of the patient with a bevel of the needle facing away from the skin of the patient. Once placement of the needle within the vasculature has been confirmed, a clinician may temporarily occlude flow in the vasculature and withdraw the needle, leaving the PIVC in place for future fluid infusion and/or blood withdrawal.

One known IV catheter system is the Saf-T-Intima™ Safety Integrated IV catheter system from Becton, Dickinson and Company (“BD”), which is shown in FIG. 1. The BD Saf-T-Intima™ catheter system 10 includes a catheter hub 12 having flexible wings 14, with a catheter 16 secured to the catheter hub 12 and extending out distally therefrom. An extension tube 18 is coupled to a proximal end of the catheter hub 12, with an adapter 20 (e.g., a Y-adapter) coupled to the proximal end of the extension tube 18. A needle introducer 22 is coupled to the adapter 20, such as to a proximal port thereon that includes a needle-free connector 24, with the needle introducer generally including a needle 26, a needle hub 28, and a telescoping needle shield 30. The needle hub 28 retains the needle 26 therein, with the needle 26 extending distally therefrom so as to pass through the extension tube 18, the catheter hub 12, and into/through a lumen of catheter 12 when needle introducer 22 is coupled to the adapter 20. The needle hub 28 is configured to be grasped by a user during insertion of the needle 26 and catheter 12 into the vasculature of the patient.

In operation of the needle introducer 22, a user may pinch the flexible wings 14 together to secure the needle 26 within the catheter hub 14 (and catheter 12) and then apply a pushing force to the needle hub 28 that causes the needle 26 to pierce the skin of a patient, with a further pushing force then causing the needle 26 and the catheter 12 to be inserted into the vasculature of the patient. Upon insertion of the needle 26 and catheter 12 into the patient, the user may then pull back on the needle hub 28 to retract the needle 26 out from the catheter 12. As the needle hub 28 is pulled back, the needle shield 30 telescopes out from the hub 28 and extends in length, with continued pulling of the needle hub 28 then causing the needle hub 28 and needle shield 30 to disconnect/separate from the adapter 20 and the needle 26 to pull out from the adapter 20 through the connector 24. As the needle 26 exits the adapter 20, the telescoped needle shield 30 covers the distal tip of the needle 26, so as to prevent an accidental needle stick from occurring.

After removal of the needle introducer, the PIV C that is left in place may be used for a future fluid infusion and/or blood withdrawal, with it recognized that such fluid infusion and/or blood withdrawal may be performed via an external device or system useable with the PIVC to aid in fluid infusion and/or blood withdrawal directly from the indwelling catheter of the PIVC. For example, a blood draw device known as PIVOT from Becton, Dickinson and Company, is configured as a single-use device which temporarily attaches to a PIVC to draw a blood sample. Using an existing peripheral intravenous line as a conduit to the vasculature, the PIVO™ device advances a flexible, internal probe or flow tube through the PIVC to (or beyond) the catheter tip to collect a blood sample. This flow tube is designed to extend beyond the suboptimal draw conditions around the indwelling line to reach vein locations where blood flow is optimal for aspiration. Once blood collection is complete, the flow tube is retracted, and the device is removed from the PIVC and discarded. An example of one such blood draw device is shown and described in U.S. Pat. No. 10,300,247 B2, which is incorporated by reference herein in its entirety.

While IV catheter systems such as the BD Saf-T-Intima™ catheter system described herein are capable of use with external devices/systems that aid in fluid infusion and/or blood withdrawal directly from the indwelling catheter of the PIVC (e.g., the BD PIVO™), it is recognized that there are limitations and/or drawbacks associated with the current configuration of such catheter systems. As one example, the configuration of the catheter system (e.g., the number of connections or ports thereon) may limit the available treatment options for a patient—such as by not enabling the administering of different compatible medications at the same time and/or performing of a blood draw when required. As another example, the configuration of the catheter system (e.g., the length of extension tube), may inhibit or otherwise increase the difficulty of advancing a flow tube of a blood draw device into and through the catheter hub.

Accordingly, a need exists for a catheter system that facilitates the connection of a blood draw device thereto and enables advancement of a flow tube into the indwelling catheter. The catheter system would also provide numerous ports or connections, whereby multiple fluid delivery devices (and associated fluid infusions) could be coupled to the catheter system in addition to a blood draw device coupled thereto.

### SUMMARY OF THE INVENTION

Provided herein is a catheter system that includes a catheter comprising a catheter distal end and a catheter proximal end and a first catheter adapter comprising an adapter body and an adapter wing joined to the adapter body. The adapter body includes a distal port, a proximal port, and a side port, with the adapter body defining a lumen therein extending between the distal port and the proximal port, and wherein the catheter proximal end is secured to the distal port and the side port is in fluid communication with the lumen and comprises a luer connection. The adapter wing includes a first wing and a second wing, with the first and second wings comprising flexible wings configured to be brought into contact with each other when a pinching force is applied thereto. The catheter system also includes an extension tube comprising a distal end and a proximal end, with the distal end of the extension tube connected to the proximal port, and a second catheter adapter connected to the proximal end of the extension tube. The catheter system further includes a blood draw device coupled to the side port, the blood draw device comprising a flow tube configured to be advanced distally out from the blood draw device and through the first catheter adapter and into the catheter, to enable performing of a blood draw.

In some embodiments, the catheter system further includes a connector coupled to the side port, the connector configured to engage the luer connection of the side port and including a needle-free connection or seal therein, the blood draw device configured to couple with the connector and engage the needle-free connection or seal.

In some embodiments, the blood draw device comprises an introducer having a proximal end and a distal end and defining an inner volume configured to movably receive the flow tube, an actuator movably coupled to the introducer and configured to move relative to the introducer to move a distal end of the flow tube from a position within the introducer to a position outside the introducer housing and past a catheter distal end of the catheter, and a coupler positioned at the distal end of the introducer and configured to engage the connector, the coupler comprising a blunt cannula and a pair of locking arms.

In some embodiments, the luer connection comprises a slip septum positioned therein configured to selectively close off the lumen in the adapter body when the blood draw device is not connected to the first catheter adapter.

In some embodiments, the second catheter adapter comprises a Y-adapter including a distal port, a proximal port, and a side port, the Y-adapter defining a lumen therein extending between the distal port and the proximal port, and with the distal port configured to receive the proximal end of the extension tube.

In some embodiments, the catheter system further includes a needle introducer engageable with the second catheter adapter, the needle introducer comprising a needle hub, a needle comprising a needle distal end and a needle proximal end, with the needle proximal end secured to the needle hub and the needle distal end positionable within a catheter lumen of the catheter and with the needle passing through the extension tube and the lumen of the adapter body, and a needle shield positioned within the needle hub, the needle shield comprising a telescoping shield extendable from a collapsed configuration to an extended configuration.

In some embodiments, with the first and second wings of the adapter wing brought into contact with each other, the needle is secured in placed within the first catheter adapter and the catheter, to enable insertion of the needle distal end into a patient without movement of the needle relative to the first catheter adapter and the catheter.

In some embodiments, the needle introducer is configured to disengage the second catheter adapter when a proximally directed pulling force is applied thereto.

In some embodiments, the needle shield is in the extended configuration upon the needle introducer disengaging the second catheter adapter, with the needle shield covering the needle distal end.

Also provided herein is a catheter system that includes a catheter comprising a catheter distal end and a catheter proximal end, and a first catheter adapter comprising a bifurcation hub and an adapter wing joined to the bifurcation hub. The bifurcation hub includes a distal port, a first proximal port, and a second proximal port, wherein the catheter proximal end is secured to the distal port and extends out distally therefrom. The adapter wing includes a first wing and a second wing, wherein the first and second wings comprise flexible wings configured to be brought into contact with each other when a pinching force is applied thereto. The catheter system also includes a first extension tube comprising a distal end and a proximal end, with the distal end of the first extension tube connected to the first proximal port, and a second extension tube comprising a distal end and a proximal end, with the distal end of the second extension tube connected to the second proximal port and the proximal end of the second extension tube comprising a luer connection and a valve. The catheter system further includes a blood draw device coupled to the luer connection of the second extension tube and configured to engage the valve, the blood draw device comprising a flow tube configured to be advanced distally out from the blood draw device and through the valve, through the first catheter adapter, and into the catheter, to enable performing of a blood draw.

In some embodiments, the catheter system further includes a second catheter adapter connected to the proximal end of the first extension tube.

In some embodiments, the second catheter adapter comprises a Y-adapter including a distal port, a proximal port, and a side port, the Y-adapter defining a lumen therein extending between the distal port and the proximal port, and with the distal port configured to receive the proximal end of the first extension tube.

In some embodiments, the catheter system further includes a clamp positioned on the first extension tube and that is operable to selectively control a fluid flow through the first extension tube.

In some embodiments, the catheter system further includes a needle introducer engageable with the second catheter adapter. The needle introducer includes a needle hub, a needle comprising a needle distal end positionable within a catheter lumen of the catheter and a needle proximal end secured to the needle hub with the needle passing through the first extension tube and through the bifurcation hub, and a needle shield positioned within the needle hub, with the needle shield comprising a telescoping shield extendable from a collapsed configuration to an extended configuration.

In some embodiments, with the first and second wings of the adapter wing brought into contact with each other, the needle is secured in placed within the first catheter adapter and the catheter, to enable insertion of the needle distal end into a patient without movement of the needle relative to the first catheter adapter and the catheter.

In some embodiments, the blood draw device comprises an introducer having a proximal end and a distal end and defining an inner volume configured to movably receive the flow tube, an actuator movably coupled to the introducer and configured to move relative to the introducer to move a distal end of the flow tube from a position within the introducer to a position outside the introducer housing and past a catheter distal end of the catheter, and a coupler positioned at the distal end of the introducer that is coupled to the luer connection, the coupler comprising a blunt cannula configured to engage the valve and provide for advancement of the flow tube therethrough.

Also provided herein is a catheter system that includes a catheter comprising a catheter distal end and a catheter proximal end, and a first catheter adapter comprising an adapter body and an adapter wing joined to the adapter body. The adapter body includes a distal port and a proximal port, with the adapter body defining a lumen therein extending between the distal port and the proximal port, and wherein the catheter proximal end is secured to the distal port. The adapter wing includes a first wing and a second wing, wherein the first and second wings comprise flexible wings configured to be brought into contact with each other when a pinching force is applied thereto. The catheter system also includes an extension tube comprising a distal end and a proximal end, with the distal end of the extension tube connected to the proximal port. The catheter system also includes a second catheter adapter connected to the proximal end of the extension tube, the second catheter adapter comprising a Y-adapter including a distal port, a proximal port, and a side port, with the Y-adapter defining a lumen therein extending between the distal port and the proximal port, and with the distal port configured to receive the proximal end of the extension tube. The catheter system further includes and a blood draw device coupleable to the second catheter adapter, the blood draw device comprising a flow tube configured to be advanced distally out from the blood draw device and through the first catheter adapter, the extension tube and the first catheter adapter, and into the catheter, to enable performing of a blood draw.

In some embodiments, the catheter system further includes a connector coupled to one of the proximal port and the side port of the second catheter adapter, the connector configured including a needle-free connection or seal therein, with the blood draw device configured to couple with the connector and engage the needle-free connection or seal.

In some embodiments, the catheter system further includes an extension set coupled to one of the proximal port and the side port of the second catheter adapter, the extension set including: a second extension tube having a distal end and a proximal end, the distal end coupled to the one of the proximal port and the side port of the second catheter adapter; and one of a luer connection and a third catheter adapter coupled to the proximal end of the second extension tube.

In some embodiments, the catheter system further includes a needle introducer engageable with the second catheter adapter. The needle introducer includes a needle hub, a needle comprising a needle distal end positionable within a catheter lumen of the catheter and a needle proximal end secured to the needle hub with the needle passing through the extension tube and the lumen of the adapter body, and a needle shield positioned within the needle hub, with the needle shield comprising a telescoping shield extendable from a collapsed configuration to an extended configuration.

In some embodiments, with the first and second wings of the adapter wing brought into contact with each other, the needle is secured in placed within the first catheter adapter and the catheter, to enable insertion of the needle distal end into a patient without movement of the needle relative to the first catheter adapter and the catheter.

### BRIEF DESCRIPTION OF THE DRAWINGS

FIG. 1 is a top view of a catheter system, as known in the prior art;

FIG. 2A is a top view of a catheter system, in accordance with an aspect of the disclosure, with the catheter system in a first configuration where a needle introducer is attached for performing a catheter insertion;

FIG. 2B is a top view of the catheter system of FIG. 2A, with the catheter system in a second configuration where a blood draw device and fluid delivery device are attached for performing a blood draw and fluid delivery;

FIG. 3 is a view of the needle introducer of FIG. 2A, illustrating a sequential operation and movement of the needle introducer;

FIG. 4 is a top view of a catheter system, in accordance with another aspect of the disclosure;

FIG. 5 is a top view of a catheter system, in accordance with another aspect of the disclosure;

FIG. 6 is a top view of a catheter system, in accordance with another aspect of the disclosure; and

FIG. 7 is a top view of a catheter system, in accordance with another aspect of the disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

As used in this specification, the words “proximal” and “distal” refer to the direction closer to and away from, respectively, a user who would place the device into contact with a patient. Thus, for example, the end of a device first touching the body of the patient would be the distal end, while the opposite end of the device (e.g., the end of the device being manipulated by the user) would be the proximal end of the device.

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

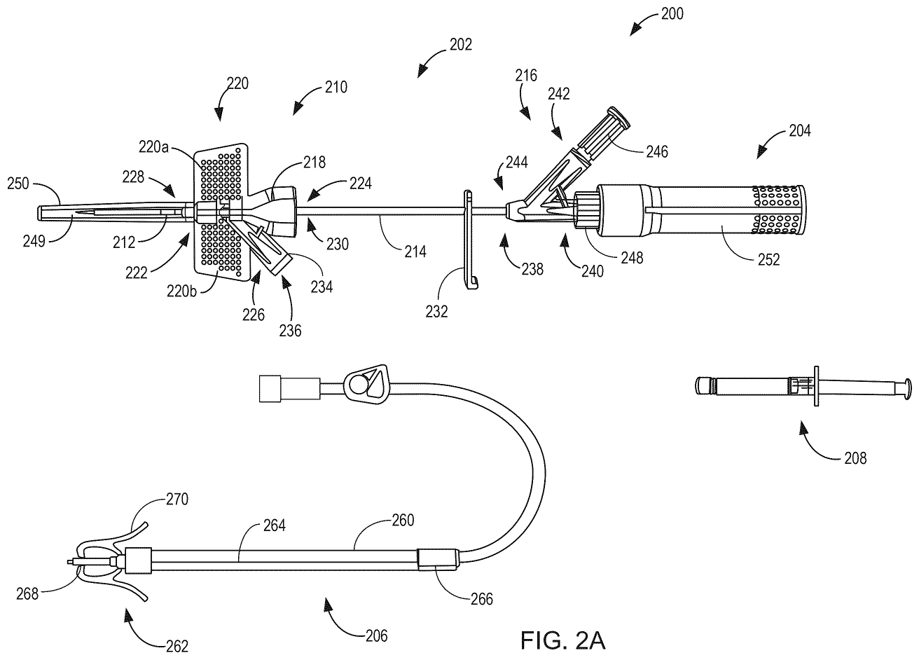

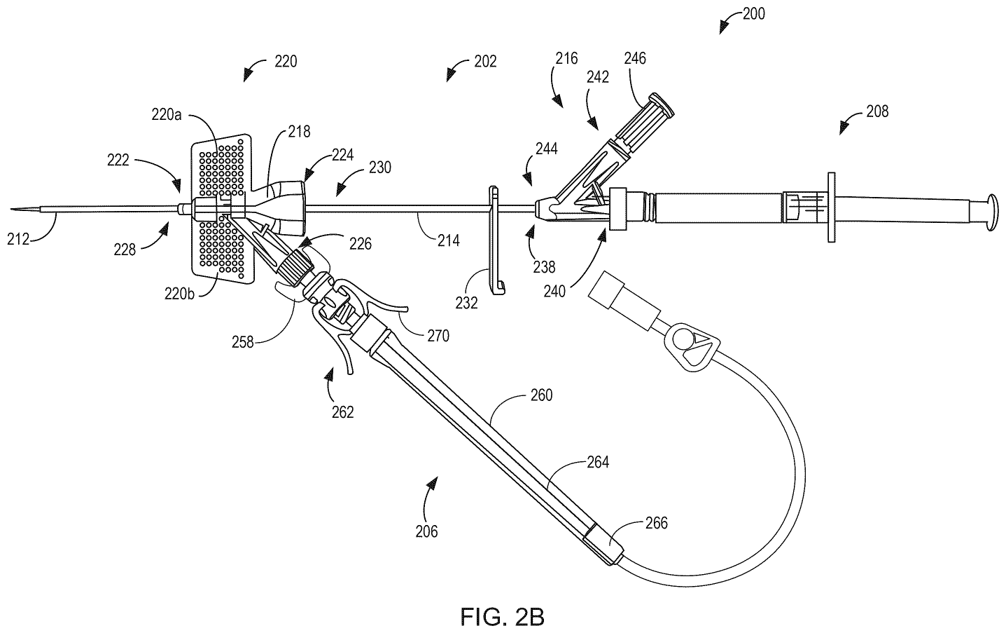

Referring to FIGS. 2A and 2B, shown is a non-limiting embodiment of a safety IV catheter system 200 (hereafter “catheter system 200”) for facilitating needle/catheter placement, blood draw and/or administering of fluids for a patient. As shown in FIGS. 2A and 2B, system 200 may include a catheter assembly 202 that provides access to the vasculature of a patient, along with a number of devices that may connected to and/or used with the catheter assembly 202 for purposes of needle/catheter placement, blood draw and/or administering of fluids. In the illustrated embodiment, devices include a needle introducer 204 (with associated safety mechanism), a blood draw device 206, and a fluid delivery device 208. As various times and stages of use, one or more of the devices may be connected to and/or used with the catheter assembly 202. For example, needle introducer 204 may be connected to catheter assembly 202 during an initial needle stick and insertion of an indwelling catheter (see FIG. 2A), with the needle introducer 204 then removed and the blood draw device 206 and fluid delivery device 208 connected to catheter assembly 202 for performing of a blood draw and/or administering of fluids (see FIG. 2B).

As shown in FIGS. 2A and 2B, the catheter assembly 202 includes a first catheter adapter 210 and associated catheter 212, along with an extension tube 214 and a second catheter adapter 216. The first catheter adapter 210 may be characterized as including an adapter body 218 and an adapter wing 220. The adapter body 218 includes a distal port 222, a proximal port 224, and a side port 226. A main lumen (not shown) is formed within the adapter body 218 that extends between the distal port 222 and the proximal port 224, and the side port 226 is in fluid communication with the main lumen via a branch lumen (not shown). The distal port 222 of adapter body 218 receives and secures a proximal end 228 of the catheter 212 therein, while the proximal port 224 receives and secures a distal end 230 of the extension tube 214 therein. In some embodiments, a clamp 232 may be provided on extension tube 214 to selectively control a flow of fluid (i.e., blood) therethrough.

According to aspects of the disclosure, the adapter wing 220 is joined to the adapter body 218, such as being formed integrally therewith as a single component. The adapter wing 220 includes a first wing 220a and a second wing 220b extending out in opposing directions from the adapter body 218. The first and second wings 220a, 220b are constructed as flexible wings that may bend and flex responsive to a pressure applied thereto. More specifically, the flexible first and second wings 220a, 220b are configured to deflect upwardly and be brought into contact with each other when a pinching force is applied thereto—with such deflection/actuation of the adapter wing 220 restricting movement of a needle within the adapter body 218, as explained in further detail below.

According to aspects of the disclosure, the side port 226 of adapter body 218 comprises a luer connection 234 thereon that provides for connection of an external device, such as blood draw device 206, to the catheter adapter 210. The side port 226 can be, for example, male or female luer locks and/or any other suitable coupler, with the non-limiting embodiment of FIG. 2A illustrating the side port 226 as a female luer connector. The side port 226 can be physically and fluidically coupled to the blood draw device 206 to enable introduction of a catheter tube into the indwelling catheter 212 and performing of a subsequent blood draw, as will be explained in further detail below. In some embodiments, a valve 236 is provided within side port 226 that functions to contain fluid (i.e., blood) within the catheter adapter 210 (i.e., within branch lumen) when no blood draw device 206 is connected thereto, by closing off the lumen. In some embodiments, the valve 236 may be provided as a slip septum, although other suitable valves may also be used.

As shown in FIG. 2A, the second catheter adapter 216 is mechanically and fluidly connected to first catheter adapter 210 via extension tube 214. In some embodiments, the second catheter adapter 216 may be constructed as a Y-adapter that includes a distal port 238, a proximal port 240, and a side port 242, with the Y-adapter defining a lumen (not shown) therein extending between the distal port 238 and the proximal port 240. The distal port 238 is configured to receive and retain a proximal end 244 of the extension tube 214. In some embodiments, and as shown in FIG. 2A, a vent plug 246 may be initially placed in the side port 242 that functions to provide venting thereto after needle/catheter insertion. Additionally, in some non-limiting embodiments or aspects, a needleless access connector 248 may be coupled to the proximal port 240 of second catheter adapter 216 to which needle introducer 204 and/or a fluid delivery device 208 may be connected. The needleless access connector 248 may be configured as a split-septum connector or self-healing septum connector, as examples.

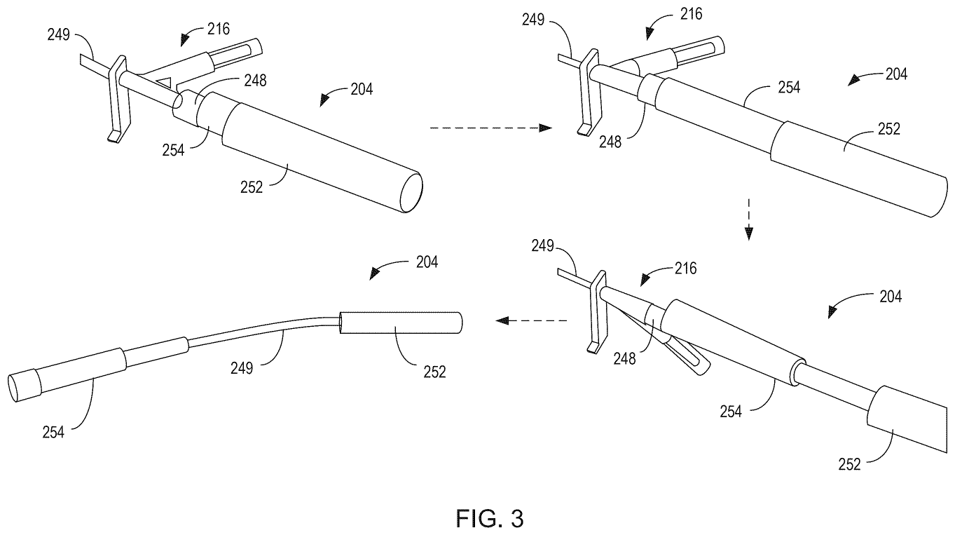

As indicated above, a needle introducer 204 is included in catheter system 200 in order to provide for an initial needle stick and insertion of catheter into the patient. As shown in FIG. 2A and now also in FIG. 3, the needle introducer 204 is coupled to the second catheter adapter 216, such as to the proximal port 240 thereof that includes needle-free connector 248, with the needle introducer 204 generally including a needle 249 (and needle tip guard 250), a needle hub 252, and a telescoping needle shield 254. The needle hub 252 retains the needle 249 therein, with the needle 249 extending distally therefrom so as to pass through the second catheter adapter 216, the extension tube 214, the first catheter adapter 210, and into/through a lumen of catheter 212 when needle introducer 204 is coupled to the second catheter adapter 216. The needle hub 252 is configured to be grasped by a user during insertion of the needle 249 and catheter 212 into the vasculature of the patient.

In operation of the needle introducer 204, with the needle 249 at an initial position within and extending out from first catheter adapter 210, a user may pinch the flexible wings 220a, 220b together to secure the needle 249 within the first catheter adapter 210 (and catheter 212) and then apply a pushing force to the needle hub 252 that causes the needle 249 to pierce the skin of a patient, with a further pushing force then causing the needle 249 and the catheter 212 to be inserted into the vasculature of the patient. As shown in FIG. 3, upon insertion of the needle 249 and catheter 212 into the patient, the user may then pull back on the needle hub 252 to retract the needle 249 out from the catheter 212. As the needle hub 252 is pulled back, the needle shield 254 telescopes out from the hub 252 and extends in length, with continued pulling of the needle hub 252 then causing the needle hub 252 and needle shield 254 to disconnect/separate from the second catheter adapter 216 (i.e., from proximal port 240) and the needle 249 to pull out from the second catheter adapter 216 through the needleless access connector 248. As the needle 249 exits the second catheter adapter 216, the telescoped needle shield 254 covers the distal tip of the needle 249, so as to prevent an accidental needle stick from occurring.

Referring now to FIG. 2B, upon insertion of the catheter 212 and removal of the needle introducer 204 from the catheter assembly 202, components such as the blood draw device 206 and one or more fluid delivery devices 208 may be connected to the catheter assembly 202. The blood draw device 206 may be connected to the side port 226 of first catheter adapter 210, while one or more fluid delivery devices 208 may be connected to the side port 242 and/or proximal port 240 of the second catheter adapter 216 (after removal of vent plug 246 and/or connector 248). In some embodiments, a connector 258 may be coupled to the side port 226 of first catheter adapter 210 to which the blood draw device 206 may be connected. The connector 258 may include a needle-free connector therein (e.g., a split-septum connector or self-healing septum connector) to facilitate connection of the blood draw device 206 thereto.

According to aspects of the disclosure, blood draw device 206 may be operated to introduce flow tube through the first catheter adapter 210 and into the vasculature of the patient, with the flow tube enabling a blood draw. As shown in FIG. 2B according to a non-limiting embodiment, the blood draw device 206 includes at least a housing 260, a coupling device or “lock” 262, a flow tube 264, and an actuator 266. As will be described in further detail below, the flow tube 264 is moveable within the housing 260 so as to provide for advancement thereof from a first or retracted position inside the housing 260 to a second or advanced position outside of the housing 260, such that a distal end thereof may be routed into the catheter assembly 202 and into or out past the indwelling catheter 212.

As shown in FIGS. 2A and 2B, the lock 262 of the instrument delivery device 200 is configured to couple the blood draw device 206 to the first catheter adapter 210. In some embodiments, the lock 262 has a blunt cannula 268 and a pair of locking arms 270. In addition, the lock 262 defines a lumen (not shown) extending therethrough The blunt cannula 268 is disposed between the pair of locking arms 270 and may have a length that is sufficient to extend through at least a portion of the connector 258 (i.e., through the needle-free connector therein).

As described above, at least a portion of the flow tube 264 is movably disposed within the housing 260. A proximal end of the flow tube 264 is coupled to the actuator 266 such that, when the actuator 266 is moved relative to the housing 260, to flow tube 264 may be moved between a first position, in which the flow tube 264 is disposed within the housing 260 (e.g., the entire flow tube 264 is disposed within the housing 260 or within the housing 260 and the lock 262) and a second position, in which the distal end portion of the flow tube 264 is at least partially disposed in a position distal to the lock 262 and distal to the first catheter adapter 210 (e.g., within catheter 212 or out past a distal tip of catheter 212).

According to aspects of the disclosure, the fluid delivery device(s) 208 connected to second catheter adapter 216 may be operated to dispense/inject a fluid into the vasculature of the patient. As shown in FIG. 2B, according to a non-limiting embodiment, fluid delivery device(s) 208 are provided as pre-filled syringe(s) (“syringe(s) 208). The syringe(s) 208 may include a luer connection at the distal tip thereof that is configured to mate with a corresponding luer connection of the second catheter adapter 216 (e.g., luer connection on side port 242). With a plunger of the syringe 208 in an initial partially retracted position, the syringe 208 may be actuated to force a medicament out from the syringe 208 and through the catheter assembly 202 for delivery to the patient.

Beneficially, the configuration of the first catheter adapter 210 and the second catheter adapter 216 allows for the connection of multiple external devices to the catheter assembly 202 at the same time. That is, the side port 226 on first catheter adapter 210 provides a dedicated port for connection of a blood draw device 206 thereto for collection of a blood sample from a patient, while the side and proximal ports 242, 240 on second catheter adapter 216 remain available for connection of one or multiple fluid delivery devices 208 thereto. The multiple ports available on the second catheter adapter 216 allow a clinician to deliver two different compatible medications at the same time, as may be desirable for treatment of a patient, and the blood draw device 206 may also be connected to the first catheter adapter 210 while the fluid delivery devices 208 are connected to second catheter adapter 216.

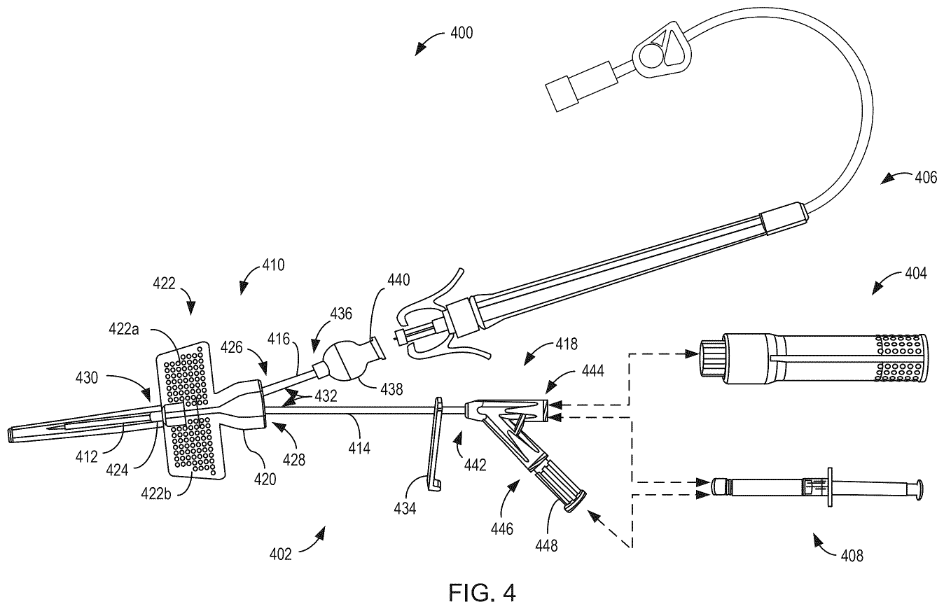

Referring now to FIG. 4, shown is another non-limiting embodiment of a catheter system 400 for facilitating needle/catheter placement, blood draw and/or administering of fluids for a patient. As shown in FIG. 4, system 400 may include a catheter assembly 402 that provides access to the vasculature of a patient, along with a number of devices that may connected to and/or used with the catheter assembly 402 for purposes of needle/catheter placement, blood draw and/or administering of fluids. In the illustrated embodiment, devices include a needle introducer 404 (with associated safety mechanism), a blood draw device 406, and one or more fluid delivery device(s) 408. As various times and stages of use, one or more of the devices may be connected to and/or used with the catheter assembly 402. For example, needle introducer 404 may be connected to catheter assembly 402 during an initial needle stick and insertion of an indwelling catheter, with the needle introducer 404 then removed and the blood draw device 406 and fluid delivery device(s) 408 connected to catheter assembly 402 for performing of a blood draw and/or administering of fluids (see FIG. 2B).

As shown in FIG. 4, the catheter assembly 402 includes a first catheter adapter 410 and associated catheter 412, along with first and second extension tubes 414, 416 and a second catheter adapter 418. The first catheter adapter 410 may be characterized as including a bifurcation hub 420 and an adapter wing 422. The bifurcation hub 420 includes a distal port 424, a first proximal port 426, and a second proximal port 428. A pair of lumens (not shown) is formed within the bifurcation hub 420 that extend between the distal port 424 and the first and second proximal ports 426, 428. The distal port 424 of bifurcation hub 420 receives and secures a proximal end 430 of the catheter 412 therein, while the proximal ports 426, 428 receive and secure distal ends 432 of the first and second extension tubes 414, 416 therein-with the first and second extension tubes 414, 416 operably connected, via the bifurcation hub 420, to a corresponding number of lumens defined by the catheter tube 12. In some embodiments, a clamp 434 may be provided on at least one of the extension tubes 414, 416 (e.g., first extension tube 414) to selectively control a flow of fluid (i.e., blood) therethrough. In some embodiments, the second extension tube 416 may be shorter in length than first extension tube 414, so as to minimize a length by which flow tube (of blood draw device 406) needs to be advanced to be routed into/through the indwelling catheter 412.

According to aspects of the disclosure, the adapter wing 422 is joined to the bifurcation hub 420, such as being formed integrally therewith as a single component. The adapter wing 422 includes a first wing 422a and a second wing 422b extending out in opposing directions from the bifurcation hub 420. The first and second wings 422a, 422b are constructed as flexible wings that may bend and flex responsive to a pressure applied thereto. More specifically, the flexible first and second wings 422a, 422b are configured to deflect upwardly and be brought into contact with each other when a pinching force is applied thereto—with such deflection/actuation of the adapter wing 422 restricting movement of a needle within bifurcation hub 420, as explained in further detail below.

According to aspects of the disclosure, a proximal end 436 of the second extension tube 416 may include a valve 438 and luer connection 440 provided thereon that provide for fluid flow control through second extension tube 416 and for connection of an external device, such as blood draw device 406, to the second extension tube 416. The luer connection 440 can be, for example, a female luer connector, as shown in FIG. 4. The valve 438 can be, for example, a SOLO™2 Valve from Becton, Dickinson and Company, or another suitable valve that closes off the proximal end 436 of second extension tube 416 and that may open responsive to application of a positive pressure or external force (e.g., a blunted cannula of blood draw device 406 being advanced therethrough) thereto. The luer connection 440 can be physically and fluidically coupled to the blood draw device 406 to enable introduction of a catheter tube into the indwelling catheter 412 and performing of a subsequent blood draw, as will be explained in further detail below.

As shown in FIG. 4, the second catheter adapter 418 is mechanically and fluidly connected to first catheter adapter 410 via second extension tube 416. In some embodiments, the second catheter adapter 418 may be constructed as a Y-adapter that includes a distal port 442, a proximal port 444, and a side port 446, with the Y-adapter defining a lumen therein extending between the distal port 442 and the proximal port 444. The distal port 442 is configured to receive and retain the proximal end 236 of the first extension tube 414. In some embodiments, and as shown in FIG. 4, a vent plug 448 may be initially placed in the side port 446 that functions to provide venting thereto after needle/catheter insertion. Additionally, in some non-limiting embodiments or aspects, a needleless access connector (not shown) may be coupled to the proximal port 444 of second catheter adapter 418 to which needle introducer 404 and/or a fluid delivery device 408 may be connected. The needleless access connector may be configured as a split-septum connector or self-healing septum connector, as examples.

Similar to as described above for FIGS. 2A, 2B and 3, a needle introducer 404 may be coupled to the second catheter adapter 418, such as to the proximal port 444 thereof, with the needle introducer 404 being operable to pierce the skin of a patient with the needle and insert catheter 12 into the vasculature of the patient. As previously described, with a needle at an initial position within and extending out from first catheter adapter 410, a user may pinch the flexible wings 422a, 422b together to secure the needle within the first catheter adapter 410 (and catheter 412) and then apply a pushing force to a needle hub that causes the needle to pierce the skin of a patient, with a further pushing force then causing the needle and the catheter 412 to be inserted into the vasculature of the patient. After placement of the indwelling catheter into the vasculature of the patient, the needle introducer 404 may then be removed from second catheter adapter 418.

As described above for FIGS. 2A, 2B and 3, upon insertion of the catheter 412 and removal of the needle introducer 404 from the catheter assembly 402, components such as the blood draw device 406 and one or more fluid delivery devices 408 may be connected to the catheter assembly 402. The blood draw device 406 may be connected to the proximal end 436 of second extension tube 416 (via luer connection 440), while one or more fluid delivery devices 208 may be connected to the side port 446 and/or proximal port 444 of the second catheter adapter 418 (after removal of vent plug 448 and/or a connector therefrom). The blood draw device 206 and fluid delivery device(s) 208 may be structured as previously described, and operable to facilitate a blood collection from a patient and/or administering of fluids to the patient.

Beneficially, the configuration of the catheter assembly 402—including a bifurcated first catheter adapter 410, a second catheter adapter 418, a pair of extension tubes 414, 416, and a second catheter adapter 418—allows for the connection of multiple external devices to the catheter assembly 402 at the same time. That is, the luer connection 440 at the proximal end 436 of second extension tube 416 provides a dedicated connector for coupling of a blood draw device 206 thereto for collection of a blood sample from a patient, while the side and proximal ports 446, 444 on second catheter adapter 418 remain available for connection of one or multiple fluid delivery devices 208 thereto. The multiple ports available on the second catheter adapter 418 allow a clinician to deliver two different compatible medications at the same time, as may be desirable for treatment of a patient, and the blood draw device 206 may also be connected while the fluid delivery devices 208 are connected to second catheter adapter 418.

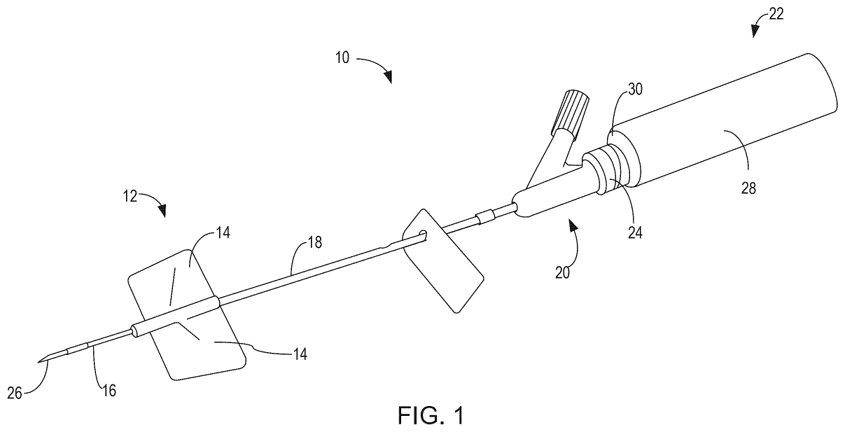

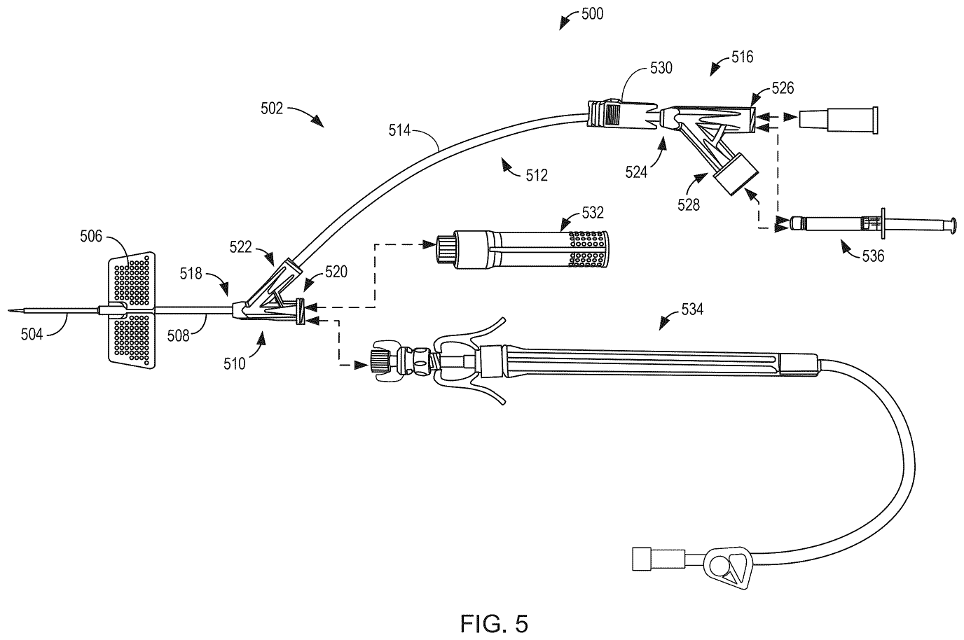

Referring now to FIGS. 5-7, additional embodiments of catheter systems 500, 600, 700 for facilitating needle/catheter placement, blood draw and/or administering of fluids for a patient are shown, according to further aspects of the disclosure. In each of the systems 500, 600, 700 of FIGS. 5-7, a catheter assembly 502, 602, 702 is provided that includes a catheter 504, 604, 704, a flexible wing catheter hub 506, 606, 706, an extension tube 508, 608, 708, and a catheter adapter 510, 610, 710. As compared to known catheter systems, such as the catheter system 10 shown and described in FIG. 1 for example, the catheter systems 500, 600, 700 of FIGS. 5-7 provide an optimized catheter assembly that is more compatible with a blood draw device and that allows for the connection of multiple external devices to the catheter assembly at the same time.

As shown in each of FIGS. 5-7, the catheter assembly 502, 602, 702 is configured such that the extension tube 508, 608, 708 thereof has an ideal length that allows for improved routing and advancement of flow tube from a blood draw device through the catheter adapter 510, 610, 710 and the extension tube 508, 608, 708 and into the indwelling catheter 504, 604, 704. According to embodiments of the disclosure, the extension tube 508, 608, 708 may be constructed to have a length so as to minimize a length by which flow tube (of a blood draw device) needs to be advanced to be routed into/through the indwelling catheter 504, 604, 704. Additionally, a plurality of ports or connections are provided on the catheter assembly 502, 602, 702 that allows for the connection of multiple external devices to the catheter assembly 502, 602, 702 at the same time—such as a blood draw device and one or more fluid delivery devices—such that a clinician may deliver two different compatible medications at the same time and also have a blood draw device connected to enable collection of a blood sample at a desired time.

Referring to FIG. 5, a catheter system 500 is provided that includes a catheter assembly 502—with the catheter assembly 502 further including a catheter 504, a flexible wing catheter hub 506, an extension tube 508, and a first catheter adapter 510. The catheter assembly 502 may also include an extension set 512 coupled to the first catheter adapter 510. The extension set 512 may include a secondary extension tube 514 and a second catheter adapter 516, with the first catheter adapter 510 connected to a distal end of the secondary extension tube 514 and the second catheter adapter 516 connected to a proximal end of the secondary extension tube 514. Each of the first catheter adapter 510 and the second catheter adapter 516 may be configured as a Y-adapter that includes a distal port, a proximal port, and a side port—i.e., a distal port 518, a proximal port 520, and a side port 522 on first catheter adapter 510 and a distal port 524, a proximal port 526, and a side port 528 on second catheter adapter 516. In some embodiments, a clamp 530 (e.g., a pinch clamp) is provided on the secondary extension tube 514 that allows for selective occlusion thereof.

The flexible wing catheter hub 506 may function similarly to the adapter wing described in the embodiments of FIGS. 2A and 2B and FIG. 4. That is, a first wing and a second wing of the flexible wing catheter hub 506 may bend and flex responsive to a pressure applied thereto. The flexible first and second wings are configured to deflect upwardly and be brought into contact with each other when a pinching force is applied thereto—with such deflection/actuation of the flexible wing catheter hub 506 restricting movement of a needle within flexible wing catheter hub 506.

According to aspects of the disclosure, the first catheter adapter 510 provides for connection of a needle introducer 532 and blood draw device 534 thereto, so as to facilitate needle/catheter placement and blood draw. Each of the needle introducer 532 and blood draw device 534 may be configured as described previously regarding the embodiments of FIGS. 2A and 2B and FIG. 4, and may be connected directly to the proximal port 520 of the first catheter adapter 510 or through a connector (e.g., needle-free connector) attached to the proximal port 520. Beneficially, attachment of the blood draw device 534 to the proximal port 520 of first catheter adapter 510 facilitates the seamless insertion of a flow tube (from the blood draw device 534) through the first catheter adapter 510 and into the indwelling catheter 504, by providing a straight passage through which the flow tube may be advanced, which may prevent tube kinking and minimize the risk of tube damage.

According to aspects of the disclosure, the extension set 512 of catheter assembly 502 may be used to provide a fluid path to catheter assembly 502 and enable connection of a fluid delivery device 536 for fluid or medication delivery. The multiple ports (i.e., side port 528 and proximal port 526) on the second catheter adapter 516 provide multiple connections for fluid delivery devices 536 to the catheter assembly 502, to enable delivery of two different compatible medications at the same time.

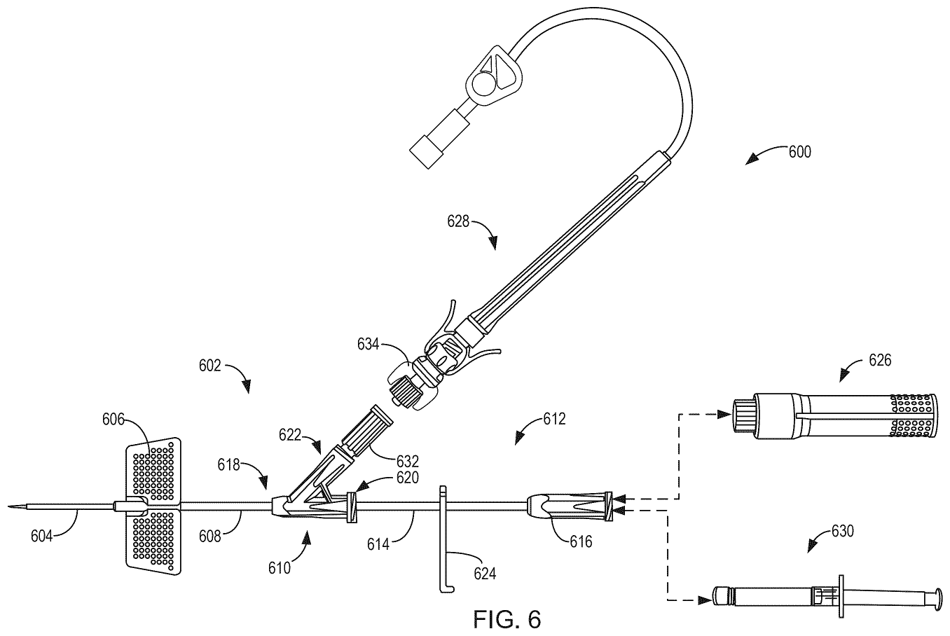

Referring to FIG. 6, a catheter system 600 is provided that includes a catheter assembly 602—with the catheter assembly 602 further including a catheter 604, a flexible wing catheter hub 606, an extension tube 608, and a catheter adapter 610. The catheter assembly 602 may also include an extension set 612 coupled to the catheter adapter 610. The extension set 612 may include a secondary extension tube 614 and a luer port 616, with the catheter adapter 610 connected to a distal end of the secondary extension tube 614 and the luer port 616 connected to a proximal end of the secondary extension tube 614. The catheter adapter 610 may be configured as a Y-adapter that includes a distal port 618, a proximal port 620, and a side port 622, with the extension set 612 connected to the proximal port 620 of the catheter adapter 610. In some embodiments, a clamp 624 (e.g., a pinch clamp) is provided on the secondary extension tube 614 that allows for selective occlusion thereof.

According to aspects of the disclosure, the catheter assembly 602 provides for connection of a needle introducer 626, blood draw device 628, and fluid delivery device 630 thereto, so as to facilitate needle/catheter placement, blood draw, and administering of a medicament. Each of the needle introducer 626 and fluid delivery device 630 may be configured as described previously regarding the embodiments of FIGS. 2A and 2B and FIG. 4, and may be connected (at different times) to the luer port 616 of extension set 612 (directly or through a connector, e.g., needle-free connector). The blood draw device 628 may be configured as described previously regarding the embodiments of FIGS. 2A and 2B and FIG. 4 and may be connected to the side port 622 of catheter adapter 610 (after removal of the illustrated vent plug 632 and attachment of a needle-free connector 634). Beneficially, attachment of the blood draw device 628 to the side port 622 of catheter adapter 610—and with a shortened extension tube 608 in catheter assembly 602—facilitates the insertion of a flow tube (from the blood draw device 628) through the catheter adapter 610 and into the indwelling catheter 604. The catheter system 600 also enables simultaneous connection of the blood draw device 628 and the fluid delivery device 630 to the catheter assembly 602.

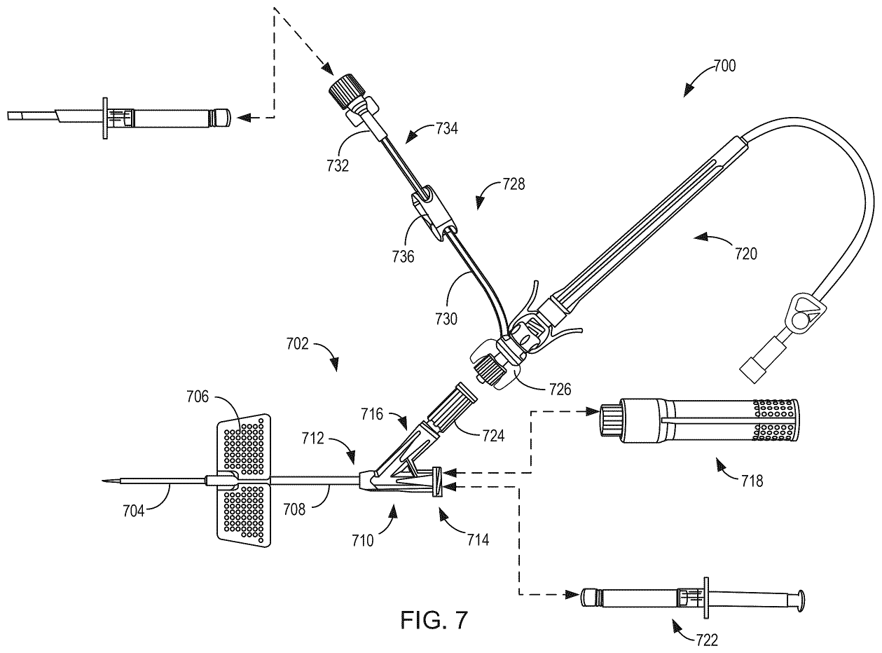

Referring to FIG. 7, a catheter system 700 is provided that includes a catheter assembly 702—with the catheter assembly 702 further including a catheter 704, a flexible wing catheter hub 706, an extension tube 708, and a catheter adapter 710. The catheter adapter 710 may be configured as a Y-adapter that includes a distal port 712, a proximal port 714, and a side port 716.

According to aspects of the disclosure, the catheter assembly 702 provides for connection of a needle introducer 718, blood draw device 720, and fluid delivery device 722 thereto, so as to facilitate needle/catheter placement, blood draw, and administering of a medicament. The needle introducer 718 and fluid delivery device 722 may be configured as described previously regarding the embodiments of FIGS. 2A and 2B and FIG. 4, and each may be connected (at different times) to the proximal port 714 of the catheter adapter 710 (directly or through a connector, e.g., needle-free connector). The blood draw device 720 may be configured as described previously regarding the embodiments of FIGS. 2A and 2B and FIG. 4 and may be connected to the side port 716 of catheter adapter 710 (after removal of the illustrated vent plug 724), with such connection provided through a connector 726. According to the illustrated embodiment, the connector 726 is configured as a t-connector (e.g., one side port arranged at a 80 degree angle relative to a longitudinal axis of connector 726), and includes a needle-free connector at the proximal end thereof, with the needle-free connector configured as a split-septum connector or self-healing septum connector, as examples. In some embodiments, an extension set 728 may be integrated with the connector 726, with the extension set 728 including a secondary extension tube 730 and a luer port 732 provided at the proximal end 734 of the secondary extension tube 730. In some embodiments, a clamp 736 (e.g., a pinch clamp) is provided on the secondary extension tube 730 that allows for selective occlusion thereof. Another fluid delivery device 722 may be connected to the luer port 732 of extension set 728 (directly or through a connector, e.g., needle-free connector).

Beneficially, attachment of the blood draw device 720 to the side port 716 of catheter adapter 710—and with a shortened extension tube 708 in catheter assembly 702—facilitates the insertion of a flow tube (from the blood draw device 720) through the catheter adapter 710 and into the indwelling catheter 704. The catheter system 700 also enables simultaneous connection of the blood draw device 720 and the fluid delivery device(s) 722 to the catheter assembly 702.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A catheter system comprising:
a catheter comprising a catheter distal end and a catheter proximal end;
a first catheter adapter comprising:
an adapter body including a distal port, a proximal port, and a side port, the adapter body defining a lumen therein extending between the distal port and the proximal port, wherein the catheter proximal end is secured to the distal port, and wherein the side port is in fluid communication with the lumen and comprises a luer connection; and
an adapter wing joined to the adapter body, the adapter wing including a first wing and a second wing, wherein the first and second wings comprise flexible wings configured to be brought into contact with each other when a pinching force is applied thereto;
an extension tube comprising a distal end and a proximal end, the distal end of the extension tube connected to the proximal port;
a second catheter adapter connected to the proximal end of the extension tube; and
a blood draw device coupled to the side port, the blood draw device comprising a flow tube configured to be advanced distally out from the blood draw device and through the first catheter adapter and into the catheter, to enable performing of a blood draw.

2. The catheter system of claim 1, further comprising a connector coupled to the side port, the connector configured to engage the luer connection of the side port and including a needle-free connection or seal therein, the blood draw device configured to couple with the connector and engage the needle-free connection or seal.

3. The catheter system of claim 2, wherein the blood draw device comprises:
an introducer having a proximal end and a distal end, and defining an inner volume configured to movably receive the flow tube;
an actuator movably coupled to the introducer, the actuator configured to move relative to the introducer to move a distal end of the flow tube from a position within the introducer to a position outside the introducer housing and past a catheter distal end of the catheter; and
a coupler positioned at the distal end of the introducer and configured to engage the connector, the coupler comprising a blunt cannula and a pair of locking arms.

4. The catheter system of claim 1, wherein the luer connection comprises a slip septum positioned therein configured to selectively close off the lumen in the adapter body when the blood draw device is not connected to the first catheter adapter.

5. The catheter system of claim 1, wherein the second catheter adapter comprises a Y-adapter including a distal port, a proximal port, and a side port, the Y-adapter defining a lumen therein extending between the distal port and the proximal port, and with the distal port configured to receive the proximal end of the extension tube.

6. The catheter system of claim 1, further comprising a needle introducer engageable with the second catheter adapter, the needle introducer comprising:
a needle hub;
a needle comprising a needle distal end and a needle proximal end, the needle proximal end secured to the needle hub and the needle distal end positionable within a catheter lumen of the catheter, with the needle passing through the extension tube and the lumen of the adapter body; and
a needle shield positioned within the needle hub, the needle shield comprising a telescoping shield extendable from a collapsed configuration to an extended configuration.

7. The catheter system of claim 6, wherein with the first and second wings of the adapter wing brought into contact with each other, the needle is secured in placed within the first catheter adapter and the catheter, to enable insertion of the needle distal end into a patient without movement of the needle relative to the first catheter adapter and the catheter.

8. The catheter system of claim 6, wherein the needle introducer is configured to disengage the second catheter adapter when a proximally directed pulling force is applied thereto.

9. The catheter system of claim 8, wherein the needle shield is in the extended configuration upon the needle introducer disengaging the second catheter adapter, with the needle shield covering the needle distal end.

10. A catheter system comprising:
a catheter comprising a catheter distal end and a catheter proximal end;
a first catheter adapter comprising:
a bifurcation hub including a distal port, a first proximal port, and a second proximal port, wherein the catheter proximal end is secured to the distal port and extends out distally therefrom; and
an adapter wing joined to the bifurcation hub, the adapter wing including a first wing and a second wing, wherein the first and second wings comprise flexible wings configured to be brought into contact with each other when a pinching force is applied thereto;
a first extension tube comprising a distal end and a proximal end, the distal end of the first extension tube connected to the first proximal port;
a second extension tube comprising a distal end and a proximal end, the distal end of the second extension tube connected to the second proximal port, and wherein the proximal end of the second extension tube comprises a luer connection and a valve; and
a blood draw device coupled to the luer connection of the second extension tube and configured to engage the valve, the blood draw device comprising a flow tube configured to be advanced distally out from the blood draw device and through the valve, through the first catheter adapter, and into the catheter, to enable performing of a blood draw.

11. The catheter system of claim 10, further comprising a second catheter adapter connected to the proximal end of the first extension tube.

12. The catheter system of claim 10, wherein the second catheter adapter comprises a Y-adapter including a distal port, a proximal port, and a side port, the Y-adapter defining a lumen therein extending between the distal port and the proximal port, and with the distal port configured to receive the proximal end of the first extension tube.

13. The catheter system of claim 10, further comprising a clamp positioned on the first extension tube and that is operable to selectively control a fluid flow through the first extension tube.

14. The catheter system of claim 11, further comprising a needle introducer engageable with the second catheter adapter, the needle introducer comprising:
a needle hub;
a needle comprising a needle distal end and a needle proximal end, the needle proximal end secured to the needle hub and the needle distal end positionable within a catheter lumen of the catheter, with the needle passing through the first extension tube and through the bifurcation hub; and
a needle shield positioned within the needle hub, the needle shield comprising a telescoping shield extendable from a collapsed configuration to an extended configuration.

15. The catheter system of claim 14, wherein with the first and second wings of the adapter wing brought into contact with each other, the needle is secured in placed within the first catheter adapter and the catheter, to enable insertion of the needle distal end into a patient without movement of the needle relative to the first catheter adapter and the catheter.

16. The catheter system of claim 10, wherein the blood draw device comprises:
an introducer having a proximal end and a distal end, and defining an inner volume configured to movably receive the flow tube;
an actuator movably coupled to the introducer, the actuator configured to move relative to the introducer to move a distal end of the flow tube from a position within the introducer to a position outside the introducer housing and past a catheter distal end of the catheter; and
a coupler positioned at the distal end of the introducer that is coupled to the luer connection, the coupler comprising a blunt cannula configured to engage the valve and provide for advancement of the flow tube therethrough.

17. A catheter system comprising:
a catheter comprising a catheter distal end and a catheter proximal end;
a first catheter adapter comprising:
an adapter body including a distal port and a proximal port, the adapter body defining a lumen therein extending between the distal port and the proximal port, wherein the catheter proximal end is secured to the distal port; and
an adapter wing joined to the adapter body, the adapter wing including a first wing and a second wing, wherein the first and second wings comprise flexible wings configured to be brought into contact with each other when a pinching force is applied thereto;
an extension tube comprising a distal end and a proximal end, the distal end of the extension tube connected to the proximal port;
a second catheter adapter connected to the proximal end of the extension tube, the second catheter adapter comprising a Y-adapter including a distal port, a proximal port, and a side port, the Y-adapter defining a lumen therein extending between the distal port and the proximal port, and with the distal port configured to receive the proximal end of the extension tube; and
a blood draw device coupleable to the second catheter adapter, the blood draw device comprising a flow tube configured to be advanced distally out from the blood draw device and through the first catheter adapter, the extension tube and the first catheter adapter, and into the catheter, to enable performing of a blood draw.

18. The catheter system of claim 17, further comprising a connector coupled to one of the proximal port and the side port of the second catheter adapter, the connector configured including a needle-free connection or seal therein, with the blood draw device configured to couple with the connector and engage the needle-free connection or seal.

19. The catheter system of claim 18, further comprising an extension set coupled to one of the proximal port and the side port of the second catheter adapter, the extension set including:
a second extension tube having a distal end and a proximal end, the distal end coupled to the one of the proximal port and the side port of the second catheter adapter; and
one of a luer connection and a third catheter adapter coupled to the proximal end of the second extension tube.

20. The catheter system of claim 17, further comprising a needle introducer engageable with the second catheter adapter, the needle introducer comprising:
a needle hub;
a needle comprising a needle distal end and a needle proximal end, the needle proximal end secured to the needle hub and the needle distal end positionable within a catheter lumen of the catheter, with the needle passing through the extension tube and the lumen of the adapter body; and
a needle shield positioned within the needle hub, the needle shield comprising a telescoping shield extendable from a collapsed configuration to an extended configuration.

21. The catheter system of claim 20, wherein with the first and second wings of the adapter wing brought into contact with each other, the needle is secured in placed within the first catheter adapter and the catheter, to enable insertion of the needle distal end into a patient without movement of the needle relative to the first catheter adapter and the catheter.
